(19) United States
(12) Patent Application Publication
(10) Pub. No.: US 2020/0306145 A1
(43) Pub. Date: Oct. 1, 2020

(54) DRY TEXTURE SPRAY

(71) Applicant: Henkel AG & Co. KGaA, Duesseldorf (DE)

(72) Inventor: SUSANNE SCHMARJE, Hamburg (DE)

(73) Assignee: Henkel AG & Co. KGaA, Duesseldorf (DE)

(21) Appl. No.: 16/815,338

(22) Filed: Mar. 11, 2020

(30) Foreign Application Priority Data
Mar. 29, 2019 (DE) 10 2019 108 227.7

(51) Int. Cl. A61K 8/02; A61K 8/81; A61K 8/25; A61K 8/73; A61K 8/41; A61K 8/34; A61Q 5/06; A61Q 5/12

(52) U.S. Cl. CPC A61K 8/027; A61K 8/8182; A61K 8/25; A61K 8/732; A61Q 5/12; A61K 8/416; A61K 8/345; A61Q 5/06; A61K 8/731

(57) ABSTRACT

The present disclosure concerns a cosmetic composition comprising a film-forming polymer, a cosmetically acceptable solvent capable of dissolving the film-forming polymer, a particulate material and a cosmetically acceptable propellant. The film-forming polymer comprises a PVP/VA co-polymer and the particulate material comprises a hydrophobized fumed silica and/or a modified starch.

### CROSS-REFERENCE TO RELATED APPLICATION

This application claims priority to German Patent Application No. 10 2019 108 227.7, filed Mar. 29, 2019, which is incorporated herein by reference in its entirety.

### TECHNICAL FIELD

The present disclosure concerns a cosmetic composition for a dry texture spray and a spray containing this composition. Such a spray is suitable for treating keratinous fibers and it is particularly suitable for treating human hair. Compared to a classic fixing spray or hair spray, this dry texture spray has no sticky phase, can be easily worked into the hair with the hands and the hair can be modeled to create textured hair bundles or textures. With the dry texture spray as contemplated herein, creative, natural and new styling trends can be easily remodeled or reworked again and again. This is also possible in the so-called layering process, i.e. with repeated spraying over the style. The spray is mainly provided as propellant-containing aerosol spray, although other forms are also possible.

### BACKGROUND

A classic fixing hairspray fixes the hair in a way that makes further modeling, after the adhesive phase is complete, impossible. Here, modeling is only possible during the bonding phase. Afterwards, individual hairs are fixed together by the spray. The resulting hairstyle appears relatively static and not very playful. Younger users in particular often want alternative products to express their attitude to life with a casual look or style.

They want products that allow a fluffy, non-fixed styling. In contrast to more classic products, a matting effect is also desired. Some users also find it unpleasant that a classic hairspray feels wet and sticky during the glue phase. Also considered desirable is the addition of body, volume, texture, feel and natural shine and, if necessary, the slight cooling effect of a propellant.

It has become undesirable to use aluminum in cosmetic products because of the possibility of health hazards, warning against excessive aluminum absorption into the human body. Aluminum, especially from aluminum salts, can be absorbed through the skin, sometimes with considerably better bioavailability than aluminum inevitably contained in food. For this reason, even some rather conservative officials or organizations dedicated to public health advise against the use of aluminum-containing cosmetics.

No known or suspected health hazard, but an undesirable effect from an aesthetic point of view can be caused by hair care products containing calcium. Even the relatively small amounts of calcium contained in tap water are such a nuisance for some people that it is countered by using rainwater, distilled water or soft bottled drinking water for washing and rinsing hair, and coagulants are used to remove calcium from the hair or on the hair, etc. The reason is that calcium has a reputation of dulling the hair in the long term and impairing its elasticity. Even if some users appreciate a matting effect of a hair care product, it is usually desired to be able to remove this matting by brushing or washing and thus restore the shine of the hair. For these reasons the use of calcium in hair care products is not pleasant for all users. For the reasons mentioned above, particulate materials such as zeolites, i.e. aluminum silicates, or calcium carbonate, as well as other calcium salts, have disadvantages with regard to their use in hair care products.

Accordingly, it is desirable to provide a cosmetic composition for treating hair that does not cause a wet or sticky feeling to occur on the hair or even skin, for example, on the hands, which come into contact with the composition. In addition, it is desirable to provide a cosmetic composition for treating hair that does not contain calcium, aluminum or zeolites. Furthermore, other desirable features and characteristics will become apparent from the subsequent detailed description and the appended claims, taken in conjunction with the accompanying drawings and the foregoing technical field and background.

### BRIEF SUMMARY

In accordance with an exemplary embodiment, a cosmetic composition is provided. The cosmetic composition comprises a film-forming polymer, a cosmetically acceptable solvent capable of dissolving the film-forming polymer, a particulate material, and a cosmetically acceptable propellant. The film-forming polymer comprises a PVP/VA co-polymer and the particulate material comprises a hydrophobized fumed silica, a modified starch, or a combination thereof.

In accordance with another exemplary embodiment, a cosmetic composition is provided. The cosmetic composition comprises a film-forming polymer, a cosmetically acceptable solvent capable of dissolving the film-forming polymer, and a particulate material. The film-forming polymer comprises a PVP/VA co-polymer and the particulate material comprises a hydrophobized fumed silica, a modified starch, or a combination thereof.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

According to an exemplary embodiment, a cosmetic composition is provided that comprises a film-forming polymer, a cosmetically acceptable solvent capable of dissolving the film-forming polymer, a particulate material and a cosmetically acceptable propellant, wherein the film-forming polymer is a PVP/VA co-polymer and the particulate material comprises a hydrophobized fumed silica, a modified starch, or a combination thereof. The cosmetic compositions as contemplated herein shall thus allow, without fixing hairs together, a loose fuzzy style, possibly with a certain matting, which can be easily created with the hands, giving fullness, volume, texture, feel and natural shine and, if necessary, a slight cooling effect by the propellant. The cosmetic composition is used for treating hair without a wet or sticky feeling occurring on the hair or even skin, for example on the hands, which come into contact with the composition. This is known as an “instant dry” effect.

A wide range of film-forming polymers can be used in cosmetic products, especially in hair care products. PVP/VA co-polymers, i.e. polyvinyl pyrrolidone/vinyl acetate co-polymers, have proved to be suitable for the styling properties intended for the cosmetic composition as contemplated herein. These have the ability to create a transparent, flexible, oxygen-permeable film on various materials, e.g. hair, which can give hair support without sticking them together. Polyvinyl pyrrolidone/vinyl acetate co-polymers are linear polymers with statistical distribution, which are produced from the monomers by radical polymerization.

The composition as contemplated herein contains a cosmetically acceptable solvent suitable for the polymer, i.e. a solvent which is considered harmless for human application and which is capable of dissolving PVP/VA co-polymers. These requirements are met, for example, by ethanol or mixtures of ethanol and water. Ethanol or mixtures of ethanol and water can be used as the solvent as contemplated herein. Water can be added to the cosmetic composition with non-anhydrous ethanol, for example about 96% ethanol, or another non-anhydrous ingredient and/or added separately. Additionally, the solvent may be denatured, for example with t-butanol and Bitrex, i.e. denatonium benzoate. This is acceptable for cosmetic purposes and prevents misuse of an alcoholic solvent as well as accidental ingestion of the cosmetic composition, for example by children. In addition to its main function of dissolving the polymer and other ingredients, the solvent can also provide a pleasant sensation due to the cooling effect of its evaporation.

Even if particulate materials have already been used in hair care products, as described above, common materials are associated with not insignificant disadvantages. For the compositions contemplated herein, experiments were carried out with harmless materials. Hydrophobized fumed silica and modified starch have proven to be suitable. These can be used as alternatives to each other or together. Additional particulate materials may also be available. The use of these particulate materials in combination with a PVP/VA co-polymer has unexpectedly proved to be particularly suitable for achieving the styling properties described at the beginning, i.e. for creating creative, natural looks, for example, by playfully remodeling or reworking them again and again. One example of hydrophobized fumed silica suitable for use in the cosmetic composition contemplated herein is silica dimethyl silylate, for example Aerosil R 972, commercially available from Evonik. An example of a modified starch suitable for use in the cosmetic composition contemplated herein is RICE PO4 NATURAL, a phosphate cross-linked starch, commercially available from Argana Starke.

While an exemplary embodiment herein contemplates the cosmetic composition without a propellant and contemplates a pump spray with the composition, the form of sprayable hair care or styling products prevailing on the market is a propellant-containing, i.e. an aerosol spray. Compared to a pump sprayer, this usually results in a finer spray pattern and the coolness caused by the evaporation of the propellant can be perceived as pleasant. A suitable propellant for use in a cosmetic composition contemplated herein is dimethyl ether.

In an exemplary embodiment, the cosmetic composition comprises as a film-forming polymer the PVP/VA co-polymer 30/70 E, i.e. a polymer of about 30% by weight vinyl pyrrolidone and about 70% by weight vinyl acetate. In an exemplary embodiment, the weight proportion of the polymer in the cosmetic composition ranges from about 0.5% to about 15%, for example, from about 2% to about 10%, such as from about 4% to about 8%. With this polymer and in these concentration ranges, the beneficial effects of the cosmetic composition described above can be achieved to an increasingly higher extent.

In an exemplary embodiment, the particulate material of the cosmetic composition contemplated herein further comprises as a modified starch a rice-based cross-linked starch phosphate ester, also referred to as a phosphate cross-linked starch, for example commercially available from Argana Starke as RICE PO4 NATURAL. In an exemplary embodiment, the weight proportion of the rice-based cross-linked starch phosphate ester in the cosmetic composition is from about 0.1% to about 10%, for example from about 0.5% to about 6%, such as from about 1% to about 2%. Rice-based means that the starch is derived from rice. With this modified starch and in these concentration ranges, the advantageous effects of the cosmetic composition described above can be achieved to an increasingly higher extent. It should be noted that a phosphate-cross-linked starch can give an aesthetic silky gloss. Due to the cross-linking, phosphate-cross-linked starch does not swell, so that it does not lead to an undesired clogging or widening of pores upon skin contact.

Furthermore, in an exemplary embodiment, the particulate material optionally additionally comprises natural cellulose fibers, for example of bamboo, or native tapioca starch. In an exemplary embodiment, the proportion by weight of the natural cellulose fibers in the cosmetic composition is up to about 1%, for example from about 0.05% to about 0.5%, such as from about 0.1% to about 0.3%. In an exemplary embodiment, the proportion by weight of the native tapioca starch in the cosmetic composition is up to about 1%, for example from about 0.05% to about 0.5%, such as from about 0.1% to about 0.3%. An example of the cellulose fibers is Sensocel be 20, commercially available from CFF. An example of native tapioca starch is Tapioca Natural 9096, commercially available from Argana Starke. With these particulate materials and in these concentration ranges, the beneficial effects of the cosmetic composition described above can be achieved to an increasingly greater extent. It should be noted that natural cellulose fibers have a matting effect and add volume. Native tapioca starch has a matting effect and gives a silky sheen. It should be noted, however, that, as has already been shown, slight matting and a silky, natural gloss are not fundamental opposites but rather different nuances within a spectrum ranging from high gloss to completely matted.

In an optional exemplary embodiment, the cosmetic composition of the present disclosure comprises a conditioning agent. An example of such a conditioning agent is a quaternary ammonium compound, such as, polyquaternium-11. In an exemplary embodiment, if the conditioning agent is included, the weight proportion of the conditioning agent in the cosmetic composition is up to about 2%, for example, from about 0.5% to about 1.8%, such as from about 1 to about 1.6%. The conditioning agent makes human hair, especially when wet, easier to comb and softer, thereby facilitating hair styling. Polyquaternium-11, commercially available for example as Gafquat 440, especially in the concentrations mentioned, has proved to be particularly suitable for inducing these effects.

The particulate material comprises as hydrophobized silicic acid, silica dimethyl silylate, in an optional exemplary embodiment. The weight proportion of the silica dimethyl silylate in the cosmetic composition contemplated herein is from about 0.1% to about 1%, for example from about 0.5% to about 0.9%, such as from about 0.7% to about 0.8%. With a particulate material comprising silica dimethyl silylate, and in the indicated concentrations, the aforementioned effects desired in the context of the cosmetic composition can be achieved particularly well and particularly markedly.

In another exemplary embodiment, the particulate material may also comprise talc, i.e. Mg₃Si₄O₁₀(OH)₂. In an exemplary embodiment, the proportion by weight of talc in the cosmetic composition is up to about 0.5%, for example, from about 0.05% to about 0.3%, such as from about 0.1% to about 0.2%. Talc can act as a carrier, release agent and lubricant. It supports the loose natural stability that can be given to the hair and slightly mattes. Especially in the concentrations indicated, its addition allows to achieve desirable effects in the context of the cosmetic composition to a particular extent.

In an optional embodiment, the cosmetic composition includes a moisturizing agent. An example of a suitable moisturizing agent for use in the cosmetic composition contemplated herein is glycerol. When a moisturizing agent is used, its weight proportion in the cosmetic composition is up to about 1%, for example from about 0.05% to about 0.5%, such as from about 0.1% to about 0.3%. A moisturizing agent helps to revitalize hair. It is especially helpful for hair that is dry and brittle due to hair dye or heat. Glycerol in particular works well against dryness and brittle hair that has become fragile and can cause split ends. Glycerol can prevent split ends, make combing easier, combat frizz and moisturize dry hair. Glycerol qualities commonly used in the cosmetic field, for example with a purity level of about 99.5%, are suitable as a component of the cosmetic composition as contemplated herein.

The cosmetic composition as contemplated herein may contain water. Water may be incorporated into the cosmetic composition with non-anhydrous ethanol, for example about 96% ethanol or another non-anhydrous ingredient and/or added separately. In an exemplary embodiment, if water is contained in the cosmetic composition, its proportion by weight in the cosmetic composition is up to about 30%, for example up to about 20%, such as up to about 10%. Water content helps to keep the volatile solvent content low, which is desired by some users. On the other hand, the ratio of water to other solvents cannot be changed arbitrarily in favor of water as otherwise the other ingredients of the cosmetic composition could no longer be sufficiently dissolved.

Optionally the cosmetic composition contains a perfume. Commercially available fragrance mixtures are suitable for perfuming the cosmetic composition, for example RUBY SPLASH, Nordic Harmony 761556 or Cotton Folia 611079. In an exemplary embodiment, the perfume is present in the cosmetic composition in an amount of from about 0.05 to about 0.15% by weight, based on the total weight of the cosmetic composition.

After the cosmetic composition of the present disclosure has been described, its use for treating keratinous fibers, in particular human hair, should also be mentioned as part of the present disclosure. The cosmetic composition is intended in particular for spraying on hair which can then be modeled into a hairstyle, for example by hand. For this purpose, the cosmetic composition may be packaged in standard aerosol packaging units, i.e. spray cans. Standard powder spray valves, e.g. NKRE250PRA800/184/5/8 from Coster, and standard hairspray actuator inserts, e.g. V04.1718 from Coster, are suitable for such aerosol cans containing the cosmetic composition.

Some users are critical of the use of propellants and are happy to accept the slightly different spray characteristics of a pump sprayer. In this respect, as an alternative to a propellant-containing composition, in an exemplary embodiment, a cosmetic composition as contemplated herein can also be a propellant-free cosmetic composition. In this case, essentially the proportions by weight of all the constituents of the composition relative to the total weight of the composition are in each case a factor of from about 1/0.6 to about 1/0.3 higher than in the case of the propellant-containing composition described above, which results from calculating out the propellant proportion, the respective upper limit having to be adapted, if necessary, in accordance with the laws of logic.

The ranges given elsewhere in this document apply to the propellant-containing composition. The ranges for the proportions by weight in the propellant-free composition are thus obtained by dividing the lower limit of a range of the propellant-containing composition by about 0.6 and dividing the upper limit of a range of the propellant-containing composition by about 0.3. For the solvent content, for example, 25%/0.6 41.7% and 60%/0.3≈200% are obtained, which, taking into account the other ranges, must be adjusted to about 98.9%, resulting in a range of from about 41.7% to about 98.9%. The calculations for the other components and ranges can be made in the same way.

A propellant-free composition can, for example, be combined and used with a pumping atomizer Thus, a ready-to-use configured container with a pump sprayer spray head containing the propellant-free composition is also an embodiment of the present disclosure.

The advantages of a propellant-free composition lie in the better yield, which enables the user to cover his needs for a long time from a relatively small packaging unit, as well as in the fact that a propellant is not released, which is generally regarded as advantageous in terms of air pollution control. However, in individual cases, these advantages may be associated with disadvantages elsewhere, so that propellant-free compositions should not be regarded as advantageous in principle. Rather, they should be seen as an alternative with which a product can be optimized to meet specific requirements.

### EXAMPLES

After the cosmetic composition as contemplated herein has been described in detail, Table 1 shows examples of concrete compositions. The indicated values represent weight percentage in relation to the total weight of the cosmetic composition.

TABLE 1

| | Ex. 1 | Ex. 2 | Ex. 3 | Ex. 4 | Ex. 5 | Ex. 6 | Ex. 7 |
|---|---|---|---|---|---|---|---|
| Ethanol 96% denat. | 28.90 | 28.80 | 28.70 | 28.50 | 28.50 | 31.35 | 31.35 |
| (t-butanol, | | | | | | | |
| PVP/VA co-polymer | 8.00 | 8.00 | 8.00 | 8.00 | 8.00 | 4.00 | 4.00 |
| 30/70 E | | | | | | | |
| Aerosil R 972 | 0.75 | 0.75 | — | — | — | — | — |
| Glycerol 99.5% | 0.20 | 0.20 | 0.20 | 0.20 | 0.20 | 0.20 | 0.20 |
| Perfume RUBY | 0.15 | — | — | — | — | — | — |
| SPLASH | | | | | | | |
| Dimethylether | 62.00 | 62.00 | 62.00 | 62.00 | 62.00 | 62.00 | 62.00 |
| Talc | — | 0.15 | — | — | — | — | — |
| Perfume Nordic | — | 0.10 | — | — | — | — | — |
| Harmony | | | | | | | |
| Rice PO4 Natural | — | — | 1.00 | 1.00 | 1.00 | 2.00 | 2.00 |
| Gafquat 440 | — | — | — | — | — | — | 1.50 |
| Perfume Cotton Folia | — | — | 0.10 | 0.10 | 0.10 | 0.05 | 0.05 |
| 611079 | | | | | | | |
| Sensocel bc 20 | — | — | | 0.20 | — | 0.20 | 0.20 |
| Tapioca Natural 9096 | — | — | — | — | 0.20 | 0.20 | 0.20 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A cosmetic composition comprising:
a film-forming polymer;
a cosmetically acceptable solvent capable of dissolving the film-forming polymer;
a particulate material, and
a cosmetically acceptable propellant,
wherein the film-forming polymer comprises a PVP/VA co-polymer and the particulate material comprises a hydrophobized fumed silica, a modified starch, or a combination thereof.

2. The cosmetic composition according to claim 1, wherein
the film-forming polymer comprises PVP/VA copolymer 30/70 E.

3. The cosmetic composition according to claim 1, wherein
the particulate material comprises a rice-based cross-linked starch phosphate ester.

4. The cosmetic composition according to claim 3, wherein
the particulate material further comprises natural cellulose fibers.

5. The cosmetic composition according to claim 1 comprising a quaternary ammonium compound as a conditioning agent.

6. The cosmetic composition according to claim 1, wherein
the particulate material comprises silica dimethyl silylate present in the cosmetic composition in an amount of from about 0.1% to about 1% by weight based on the total weight of the cosmetic composition.

7. The cosmetic composition according to claim 1, wherein the particulate material comprises talc present in the cosmetic composition in an amount of up to about 0.5% by weight based on the total weight of the cosmetic composition.

8. The cosmetic composition according to claim 1, further comprising glycerol as a moisturizing agent, wherein the glycerol is present in the cosmetic composition in an amount of up to about 1% by weight based on the total weight of the cosmetic composition.

9. The cosmetic composition according to claim 1 further comprising water present in the cosmetic composition in an amount of up to about 30% by weight based on the total weight of the cosmetic composition.

10. The cosmetic composition according to claim 2, wherein the PVP/VA copolymer 30/70 E is present in the cosmetic composition in an amount of from about 0.5% to about 15% by weight based on the total weight of the cosmetic composition.

11. The cosmetic composition according to claim 3, wherein the rice-based cross-linked starch phosphate ester is present in the cosmetic composition in an amount of from about 0.1% to about 10% by weight based on the total weight of the cosmetic composition.

12. The cosmetic composition according to claim 4, wherein the natural cellulose fibers comprise bamboo, the weight proportion of which in the cosmetic composition is up to about 1% based on the total weight of the cosmetic composition.

13. The cosmetic composition according to claim 4, wherein the natural cellulose fibers comprise native tapioca starch, the weight proportion of which in the cosmetic composition is up to about 1% based on the total weight of the cosmetic composition.

14. The cosmetic composition according to claim 5, wherein the quaternary ammonium compound comprises polyquaternium-11 present in the cosmetic composition in an amount of up to about 2% by weight based on the total weight of the cosmetic composition.

15. A cosmetic composition comprising:
a film-forming polymer;
a cosmetically acceptable solvent capable of dissolving the film-forming polymer; and
a particulate material,
wherein the film-forming polymer comprises a PVP/VA co-polymer and the particulate material comprises a hydrophobized fumed silica, a modified starch, or a combination thereof.

16. The cosmetic composition according to claim 15, wherein the film-forming polymer comprises PVP/VA copolymer 30/70 E.

17. The cosmetic composition according to claim 15, wherein the particulate material comprises a rice-based cross-linked starch phosphate ester.

18. The cosmetic composition according to claim 17, wherein the particulate material further comprises natural cellulose fibers.

19. The cosmetic composition according to claim 15, comprising a quaternary ammonium compound as a conditioning agent.

20. The cosmetic composition according to claim 15, further comprising glycerol as a moisturizing agent.
